(19) United States
(12) Patent Application Publication
(10) Pub. No.: US 2002/0131908 A1
(43) Pub. Date: Sep. 19, 2002

(54) Selective release apparatus for fluids

(76) Inventor: George Benda, Itasca, IL (US)

Correspondence Address: Clifford H. Kraft, 320 Robin Hill Dr., Naperville, IL, 60540, US

(21) Appl. No.: 09/753,325

(22) Filed: Dec. 30, 2000

Related U.S. Application Data
(60) Provisional application No. 60/175,841, filed on Jan. 13, 2000.

(51) Int. Cl. A61L 9/14

(52) U.S. Cl. 422/123; 422/306

(57) ABSTRACT

A selective release apparatus where an active agent can be released into a surrounding fluid like air upon detection of a contaminant compound such as an odor. The active agent can be a fragrance or a compound that combats or converts the contaminant. An exit orifice can be attached to a capsule or sack containing the active agent. A gas or compound selective polymer can surround the sack or capsule. Dilation of the polymer upon exposure to a target contaminant compound can either cause the sack or capsule to rupture releasing the active agent, or by increasing pressure in the sack or capsule, cause the active agent to be released through the exit orifice.

This application is based upon, and claims priority from, U.S. Provisional Patent Application 60/175,841 filed Jan. 13, 2000 with the same title, and this application incorporates that provisional application (60/175,841) by reference.

### BACKGROUND

1. Field of the Invention

This invention relates generally to the field of the release of compounds into air or water, and more particularly to a selective release apparatus that releases a compound upon stimulus from a gas or other compound.

2. Description of Related Art

In the field of odor removal or air purification, it is highly desirable to be able to release special agents or compounds into air or water or other fluid to either remove an odor, convert a compound, provide a fresh or perfume scent, or other purpose. Since such agents or compounds are expensive, and most devices designed to release them contain only a finite reservoir of the agent, it would be very desirable to be able to sense the presence of a specific contaminant or odor causing compound or set of compounds, and then release enough of the agent to either remove the odor, mask it, or convert it to a non-contaminant.

Prior art methods have employed various chemical or electronic sensors to determine when a contaminant is present. These methods however, usually require electrical power, electronics, processors, etc., and can be bulky and expensive. Also, they generally require a mechanical release mechanism separate from the detection mechanism.

A particular location where an agent release product is desirable is in a bathroom or lavatory area where sometimes undesirable smells or odors can be produced. It is very desirable to sense these odors when their concentration is fairly low, and release a fragrance agent into the air to either mask them or convert them to a non-odorous compounds.

What is badly needed is a very small, cheap apparatus that could be attached to a toilet, or mounted on a wall in an area likely to become contaminated with either odor causing or otherwise objectionable compounds. Such a device should detect such target compounds in a fluid like air or water and selectively release agent compounds to either combat the odor or convert the contaminants to other, less objectionable, compounds.

### SUMMARY OF THE INVENTION

The present invention relates to a selective response, controlled release mechanism for fluids such as air or water, where a gas or compound sensitive polymer expands or contracts in the presence of the undesirable contaminant, and this mechanical change in the shape or position of the polymer itself causes a release valve to open or close on a reservoir of agent that is then selectively released into the surrounding air, water, or other fluid.

The active fluid agent is contained in a capsule, a sack, or is contained in a droplet by its surface tension. The capsule, sack, or droplet is coated with a compound selective polymer. In response to its target compound, the polymer changes shape. The capsule, sack, or droplet is affected by the change in shape in a manner that results in a controlled release of the active compound into the surrounding fluid. For example, in one embodiment of the present invention, the active agent container is squeezed by expansion of the polymer upon presence of a target compound causing the active agent to be dispensed through an exit orifice.

Polymers such as poly-styrene, poly-alpha-methyl-styrene, and many others can be used in the present invention. The important characteristic of the polymer is that it change size when exposed to a target compound. Various polymer sensors are described in U.S. Pat. Nos. 5,959,191 and 5,571,401 which are hereby incorporated by reference. These sensors use the polymer's mechanical dilation to change resistance. The present invention uses the polymer's dilation mechanically to directly release active agents.

### BRIEF DESCRIPTION OF THE DRAWINGS

For a more complete understanding of this invention, reference should now be made to the embodiments illustrated in greater detail in the accompanying drawings and described below by way of examples of the invention.

FIG. 1 shows an embodiment of the present invention which uses a capsule with a release orifice.

FIG. 2 shows an embodiment of the present invention in a frame with multiple polymer sensors.

FIG. 3 shows an embodiment of the present invention that uses a an active agent encased in a polymer.

It should be understood, of course, that the invention is not necessarily limited to the particular embodiments illustrated herein.

### DESCRIPTION OF PREFERRED EMBODIMENTS

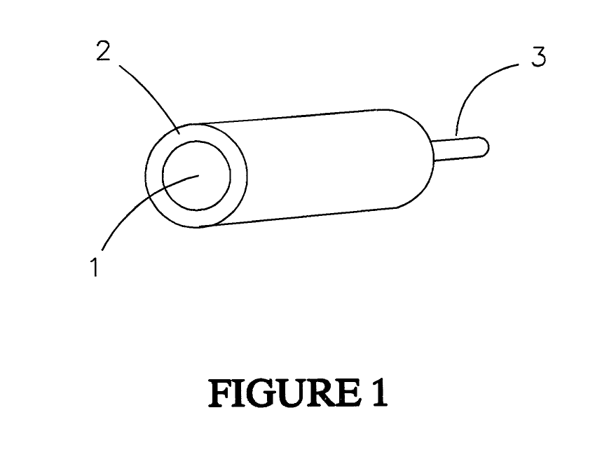

Turning to FIG. 1, a cutout view of an embodiment of the present invention can be seen that has a container 1 for active agent encased in a polymer shell 2. Also an exit orifice 3 can be seen. When the selective polymer 2 senses a target compound, it can contract around the active agent 1 increasing pressure in the agent fluid and causing it to exit through the exit orifice 3.

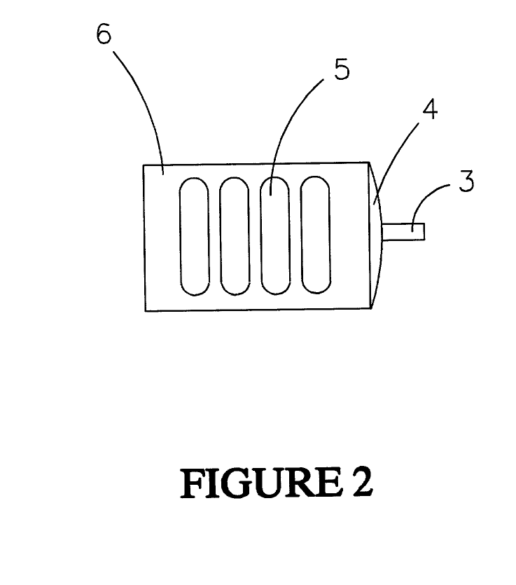

FIG. 2 shows a different embodiment of the present invention. In this embodiment, a solid frame 6 surrounds a sack or capsule 4 containing an active agent compound. An exit orifice 3 penetrates into the sack or capsule 4. Strips or other constructions of compound sensitive polymer 5 surround the sack or capsule 4 and connect to the frame 6 or simply abut the frame 6. Each strip or piece of polymer 5 can be sensitive to a different target compound. When any particular target compound is present, the polymer sensitive to it contracts of otherwise changes shape, pressing on the sack or capsule 4 of active agent causing it to be pressed out of the exit orifice 3 into the surrounding fluid (usually the air).

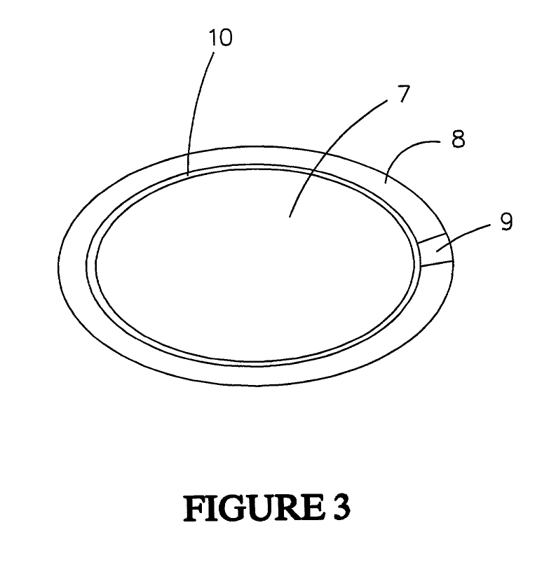

FIG. 3 shows an embodiment of the present invention that is a circular or oval capsule which has an inner compartment 7 surrounded by a shell of protective material 10 such as the

I claim:

1. An apparatus for the release of an active fluid agent comprising:
a reservoir of active agent;
a compound selective polymer in proximity to said reservoir;
a release orifice in said reservoir, said compound selective polymer experiencing a change of shape upon detection of a target compound, said change of shape exerting pressure on said reservoir causing said active agent to exit said reservoir through said release orifice.

2. The apparatus of claim 1 wherein said change of shape is an expansion.

3. The apparatus of claim 1 wherein said change of shape is a contraction.

4. The apparatus of claim 1 wherein said compound selective polymer is a polystyrene.

5. The apparatus of claim 1 wherein said compound selective polymer is a polyalphmethylstyrene.

6. An apparatus for the release of an active fluid agent comprising:
means for holding a quantity of an active fluid agent;
means for releasing said active fluid agent into a surrounding environment upon detection of presence of a predetermined target compound, said means for releasing comprising a compound selective polymer.

7. The apparatus of claim 6 wherein said means for releasing said active fluid agent further comprises an orifice.

8. The apparatus of claim 6 wherein said means for releasing said active fluid agent is a polystyrene.

9. The apparatus of claim 6 wherein said means for releasing said active fluid agent is a polyalphamethylstyrene.

10. A method for releasing a an active fluid agent into an environment upon detection of a target compound comprising the steps of:
storing an active fluid agent in polymer reservoir;
said polymer reservoir expanding or contracting in a presence of a target compound;
said expansion or contraction expelling a portion of said active fluid agent into said environment.

11. The method of claim 10 wherein said polymer is a polystyrene.

12. The method of claim 10 wherein said polymer is a polyalphamethylstyrene.

13. The method of claim 10 wherein said active agent is a perfume compound.

14. An apparatus for releasing a sweet smelling compound into surrounding air in a room where odors are generated comprising a reservoir containing a volume of the sweet smelling compound, the reservoir being made from a special polymer which detects certain order causing compounds and contracts or expands in their presence, this contraction or expansion pushing the sweet smelling compound out of a small hole in the reservoir when the one of the odder causing compounds is present.

15. The apparatus of claim 14 where the sweet smelling compound is a perfume.

16. The apparatus of claim 15 where the special polymer is a polystyrene.

17. The apparatus of claim 15 where the special polymer is a polyalphamethylstyrene.
